(19) United States
(12) Patent Application Publication
(10) Pub. No.: US 2003/0199584 A1
(43) Pub. Date: Oct. 23, 2003

(54) Reduction of hair growth

(76) Inventors: Gurpreet S. Ahluwalia, Newton, MA (US); Peter Styczynski, Wrentham, MA (US); Douglas Shander, Acton, MA (US)

Correspondence Address: FISH & RICHARDSON PC, 225 FRANKLIN ST, BOSTON, MA, 02110, US

(21) Appl. No.: 10/397,625

(22) Filed: Mar. 26, 2003

Related U.S. Application Data
(60) Provisional application No. 60/372,555, filed on Apr. 11, 2002.

(51) Int. Cl. A61K 31/202; A61K 31/198; A61K 7/15

(52) U.S. Cl. 514/560; 514/564

(57) ABSTRACT

A method of reducing hair growth includes topical application of a composition including α-difluoromethylornithine and a penetration enhancer. The penetration enhancer may be, for example, a cis-fatty acid, a terpene, a nonionic surfactant, SEPA, a film forming agent, dipropylene glycol dimethylether, cetiol, Captex-300, lauryl alcohol, triacetin, 1-dodecyl-2-pyrrolidanone, or Eston 3601.

### CROSS-REFERENCE TO RELATED APPLICATIONS

Under 35 U.S.C. § 119(e)(1), this application claims the benefit of prior U.S. provisional application 60/372,555, filed Apr. 11, 2002, hereby incorporated by reference in its entirety.

### BACKGROUND

The invention relates to reducing hair growth in mammals, particularly for cosmetic purposes.

A main function of mammalian hair is to provide environmental protection. However, that function has largely been lost in humans, in whom hair is kept or removed from various parts of the body essentially for cosmetic reasons. For example, it is generally preferred to have hair on the scalp but not on the face.

Various procedures have been employed to remove unwanted hair, including shaving, electrolysis, depilatory creams or lotions, waxing, plucking, and therapeutic antiandrogens. These conventional procedures generally have drawbacks associated with them. Shaving, for instance, can cause nicks and cuts, and can leave a perception of an increase in the rate of hair regrowth. Shaving also can leave an undesirable stubble. Electrolysis, on the other hand, can keep a treated area free of hair for prolonged periods of time, but can be expensive, painful, and sometimes leaves scarring. Depilatory creams, though very effective, typically are not recommended for frequent use due to their high irritancy potential. Waxing and plucking can cause pain, discomfort, and poor removal of short hair. Finally, antiandrogens—which have been used to treat female hirsutism—can have unwanted side effects.

It has previously been disclosed that the rate and character of hair growth can be altered by applying to the skin inhibitors of certain enzymes. These inhibitors include inhibitors of 5-alpha reductase, omithine decarboxylase, S-adenosylmethionine decarboxylase, gamma-glutamyl transpeptidase, and transglutaminase. See, for example, Breuer et al., U.S. Pat. No. 4,885,289; Shander, U.S. Pat. No. 4,720,489; Ahluwalia, U.S. Pat. No. 5,095,007; Ahluwalia et al., U.S. Pat. No. 5,096,911; and Shander et al., U.S. Pat. No. 5,132,293.

α-Difluoromethylomithine (DFMO) is an irreversible inhibitor of ornithine decarboxylase (ODC), a rate-limiting enzyme in the de novo biosynthesis of putrescine, spermidine, and spermine. The role of these polyamines in cellular proliferation is not yet well understood. However, they seem to play a role in the synthesis and/or regulation of DNA, RNA and proteins. High levels of ODC and polyamines are found in cancer and other cell types that have high proliferation rates.

DFMO binds the ODC active site as a substrate. The bound DFMO is then decarboxylated and converted to a reactive intermediate that forms a covalent bond with the enzyme, thus preventing the natural substrate omithine from binding to the enzyme. Cellular inhibition of ODC by DFMO causes a marked reduction in putrescine and spermidine and a variable reduction in spermine, depending on the length of treatment and the cell type. Generally, in order for DFMO to cause significant antiproliferative effects, the inhibition of polyamine synthesis must be maintained by continuous inhibitory levels of DFMO because the half-life of ODC is about 30 min, one of the shortest of all known enzymes.

A skin preparation containing DFMO (sold under the name Vaniqa® by Bristol Myers Squibb), has been approved by the Food and Drug Administration (FDA) for the treatment of unwanted facial hair growth in women. Its topical administration in a cream based vehicle has been shown to reduce the rate of facial hair growth in women. Vaniqa® facial cream includes a racemic mixture of the “D-” and “L-” enantiomers of DFMO (i.e., D,L-DFMO) in the monohydrochloride form at a concentration of 13.9% by weight active (15%, as monohydrochloride monohydrate). The recommended treatment regimen for Vaniqa® is twice daily. The cream base vehicle in Vaniqa® is set out in Example 1 of U.S. Pat. No. 5,648,394, which is incorporated herein by reference.

It generally takes about eight weeks of continuous treatment before the hair growth-inhibiting efficacy of Vaniqa® cream becomes apparent. Vaniqa®D cream has been shown to decrease hair growth an average of 47%. In one study, clinical successes were observed in 35% of women treated with Vaniqa® cream. These women exhibited marked improvement or complete clearance of their condition as judged by physicians scoring a decrease in visibility of facial hair and a decrease in skin darkening caused by hair. Another 35% of the women tested experienced some improvement in their condition. However, there were some women who exhibited little or no response to treatment.

The ability of hydrophilic molecules like DFMO to penetrate into the skin is restricted by the stratum comeum or outer most layer of the skin, which provides an excellent barrier against the entry of foreign substances, including drugs and chemicals into the body. The skin penetration of a compound is therefore dependent upon its physico/chemical properties as well as on the properties of the carrier vehicle. A diverse array of factors can influence penetration. The average percutaneous absorption of eflornithine (DFMO) from Vaniqa® is less than 1%.

Molecules that are identical to each other in chemical structural formula and yet are not superimposable upon each other are enantiomers. In terms of their physiochemical properties enantiomers differ only in their ability to rotate the plane of plane-polarized light, and this property is frequently used in their designation. Those entiomers that rotate plane-polarized light to the right are termed dextrorotatory, indicated by either a (+)- or d- or D- before the name of the compound; those that rotate light to the left are termed laevorotatory indicated by a (−)- or 1- or L- prefix. A racemic mixture is indicated by either a (±)- or d,l- or D,L- prefix. By another convention (or nomenclature), the R,S or the sequence rule can be used to differentiate enantiomers based on their absolute configuration. Using this system the L-DFMO corresponds to the R-DFMO, and the D-DFMO corresponds to the S-DFMO. Enantiomers are physiochemically similar in that they have similar melting points, boiling points, relative solubility, and chemical reactivity in an achiral environment. A racemate is a composite of equal molar quantities of two enantiomeric species, often referred to as the DL-form. Individual enantiomers of chiral molecules may possess different pharmacological profiles, i.e., differences in pharmacokinetics, toxicity, efficacy, etc.

### SUMMARY

The present invention provides a method (typically a cosmetic method) of reducing human hair growth by applying to the skin, in an amount effective to reduce hair growth, a dermatologically acceptable topical composition including α-difluoromethylomithine (DFMO) and a dermatologically acceptable vehicle. The vehicle includes one or more of the chemical agents (described below) that enhances the penetration of DFMO. The vehicle may include, for example, from 0.1% to 20% of a penetration enhancer by weight, preferably from 1% to 12% of the penetration enhancer by weight, more preferably from 2% to 10% of the penetration enhancer by weight, and most preferably 4% to 10% urea by weight. The unwanted hair growth may be undesirable from a cosmetic standpoint or may result, for example, from a disease or an abnormal condition (e.g., hirsutism).

For purposes of this application, the vehicle includes all components of the composition except the DFMO. DFMO, as used herein, includes DFMO itself and pharmaceutically acceptable salts thereof.

Preferably the DFMO will comprise at least about 70% or 80%, more preferably at least about 90%, most preferably at least about 95% of the L-DFMO. Ideally, the DFMO will be substantially optically pure L-DFMO. “Substantially optically pure” means that the DFMO comprises at least 98% L-DFMO. “Optically pure” L-DFMO means that the DFMO comprises essentially 100% L-DFMO.

Preferred compositions include about 0.1% to about 30%, preferably about 1% to about 20%, more preferably about 5% to about 15%, by weight of the DFMO.

The present invention also provides topical compositions including a dermatologically or cosmetically acceptable vehicle, one or more of the chemical agent(s), and difluoromethylomithine in an amount effective to reduce hair growth.

The above compositions generally have an enhanced efficacy relative to similar compositions having vehicles not containing the chemical agent(s). This enhanced efficacy can manifest itself, for example, in earlier onset of hair growth inhibiting activity, greater reduction of hair growth rate, and/or greater number of subjects demonstrating reduced hair growth.

Other features and advantages of the invention will be apparent from the description and from the claims.

### DETAILED DESCRIPTION

A preferred composition includes DFMO in an amount effective to reduce hair growth in a cosmetically and/or dermatologically acceptable vehicle including at least 1% by weight of one or more of the preferred penetration enhancer. The composition may be a solid, semi-solid, cream or liquid. The composition may be, for example, a cosmetic and dermatologic product in the form of an, for example, ointment, lotion, foam, cream, gel, or solution. The composition may also be in the form of a shaving preparation or an aftershave. The vehicle itself can be inert or it can possess cosmetic, physiological and/or pharmaceutical benefits of its own.

The composition may include one or more other types of hair growth reducing agents, such as those described in U.S. Pat. No. 5,364,885 or U.S. Pat. No. 5,652,273.

The concentration of DFMO in the composition may be varied over a wide range up to a saturated solution, preferably from 0.1% to 30% by weight; the reduction of hair growth increases as the amount of DFMO applied increases per unit area of skin. The maximum amount effectively applied is limited only by the rate at which the DFMO penetrates the skin. The effective amounts may range, for example, from 10 to 3000 micrograms or more per square centimeter of skin.

Vehicles can be formulated with liquid or solid emollients, solvents, thickeners, humectants and/or powders. Emollients include, for example, stearyl alcohol, mink oil, cetyl alcohol, oleyl alcohol, isopropyl laurate, polyethylene glycol, olive oil, petroleum jelly, palmitic acid, oleic acid, and myristyl myristate. Solvents include, for example, water, ethyl alcohol, isopropanol, acetone, diethylene glycol, ethylene glycol, dimethyl sulfoxide, and dimethyl formamide.

Optically pure L-DFMO can be prepared by known methods. See, for example, U.S. Pat. No. 4,309,442, Gao et al., Ann. Pharm. Fr. 52(4):184-203 (1994); Gao et al., Ann. Pharm. Fr. 52(5):248-59 (1994); and Jacques et al., Tetrahedron Letters, 48:4617 (1971), all of which are incorporated by reference herein.

The composition should be topically applied to a selected area of the body from which it is desired to reduce hair growth. For example, the composition can be applied to the face, particularly to the beard area of the face, i.e., the cheek, neck, upper lip, or chin. The composition also may be used as an adjunct to other methods of hair removal including shaving, waxing, mechanical epilation, chemical depilation, electrolysis and laser-assisted hair removal.

The composition can also be applied to the legs, arms, torso or armpits. The composition is particularly suitable for reducing the growth of unwanted hair in women, particularly unwanted facial hair, for example, on the upper lip or chin. The composition should be applied once or twice a day, or even more frequently, to achieve a perceived reduction in hair growth. Perception of reduced hair growth can occur as early as 24 hours or 48 hours (for instance, between normal shaving intervals) following use or can take up to, for example, three months. Reduction in hair growth is demonstrated when, for example, the rate of hair growth is slowed, the need for removal is reduced, the subject perceives less hair on the treated site, or quantitatively, when the weight of hair removed (i.e., hair mass) is reduced (quantitatively), subjects perceive a reduction, for example, in facial hair, or subjects are less concerned or bothered about their unwanted hair (e.g., facial hair).

Preparation of the DFMO Containing Formulations

Formulations were typically prepared by adding the desired amount of powdered test material to the base formulations that were similar to as described in the U.S. Pat. Nos. 5,648,394 and 5,132,293. In cases where the enhancer was in the liquid form the appropriate amount was added to give the desired final concentration and the control formulation received the same amount of water such that any dilution of the base formulation was normalized. The constituents of the two base formulations used are listed in Table 1. The cream-based formulation was used in the human clinical trials that led to its marketing approval by the FDA under the trade name, Vaniqa. Additional formulations are described in the examples.

TABLE 1
Components of the two test formulations without DFMO

| Hydrophilic Formulation 1^{a} | | Cream Formulation 1^{b} | |
|---|---|---|---|
| Water | 68% | Water | 80% |
| Ethanol | 16% | Glyceryl Stearate | 4% |
| Propylene Glycol | 5% | PEG-100 | 4% |
| Dipropylene Glycol | 5% | Cetearyl Alcohol | 3% |
| Benzyl Alcohol | 4% | Ceteareth-20 | 2.5% |
| Propylene Carbonate | 2% | Mineral Oil | 2% |
| | | Stearyl Alcohol | 2% |
| | | Dimethicone | 0.5% |
| | | Phenoxyethanol | 0.3% |
| | | Methylparaben | 0.09% |
| | | Propylparaben | 0.036% |

^{a}U.S. Pat. No. 5,132,293;
^{b}U.S. Pat. No. 5,648,394

Skin Penetration Assay (Diffusion Method)

Protocol 1

An in vitro diffusion assay was established based on that reported by Franz. Dorsal skin from Golden, Syrian hamsters or Hartley guinea pigs is clipped with electric clippers, trimmed to the appropriate size and placed in a glass diffusion chamber. The receptor fluid consisted of phosphate buffered saline, an isotonic solution for maintaining cell viability and 0.1% sodium azide, a preservative and was placed in the lower chamber of the diffusion apparatus such that the level of the receptor fluid was in parallel with the mounted skin. After equilibration at 37° C. for at least 30 minutes, 10 μl or 20 μl of the test or control formulation containing equal amounts of DFMO were added to the surface of the skin and gently spread over the entire surface with a glass stirring rod. A radiotracer amount of 14C-DFMO (0.5-1 microCurie per diffusion chamber) was used in the formulations to assess DFMO penetration. Penetration of DFMO was determined by removing an aliquot (400 μL) periodically throughout the course of the experiment, and quantitating radioactivity using liquid scintillation.

Protocol 2

This procedure is similar to that described in Protocol 1 with the exception that prior to the application of radiolabeled DFMO, the skin surfaces received 1 ml of the formulation without DFMO. After 15 minutes the formulation was removed and the surface of the skin was gently dried with a cotton swab. Radiolabeled DFMO was then applied to the skin and the experiment was completed as described in Protocol 1.

The compound DFMO used in these studies has been referred in our previous patents and literature as: 2′-alpha difluoromethyl omithine; eflornithine; eflornithine. HCL.H₂O; eflornithine.HCL. In addition, the isomers or enantiomers of DFMO can be used that include D-DFMO; L-DFMO and D,L-DFMO or S-DFMO; R-DFMO and S,R-DFMO.

Skin Penetration Enhancement Effect by Preferred Chemical Agent or Agents from a Select Chemical Class:

Several cis-fatty acids with the double bond at various positions as well as elaidic acid, the trans isomer of oleic acid were tested in compositions containing DFMO. cis-Fatty acids, and in particular oleic acid, were shown (Table 2) to increase in skin penetration whereas elaidic acid, the trans-isomer of oleic acid was devoid of DFMO penetration enhancing properties.

TABLE 2
Effect of Fatty Acids (10%) on DFMO Penetration
through Hamster Skin.

| cis-Fatty Acid | Fold Enhancement |
|---|---|
| Erucic Acid | 2.18 ± 2 |
| Palmitoleic Acid | 2.65 ± .72 |
| Petroselenic Acid | 1.40 ± 1.5 |
| Oleic Acid | 2.85 ± .76 |

Further confirmation of the cis fatty acid action on skin penetration was obtained by pretreating the skin overnight with oleic acid or elaidic acid. The following day DFMO was applied to the surface of the skin in the hydroalcoholic formulation. DFMO penetration into the receptor fluid was measured hourly over eight hours as shown in figures. Oleic acid pretreatment of the skin resulted in a 10-fold enhancement of skin penetration as shown in FIG. 1, whereas, pretreatment with elaidic acid produced no increase in skin penetration of DFMO as depicted in FIG. 2. The data indicates that the cis fatty acids act on the skin to enhance DFMO penetration, and that the cis double bond is requisite for the enhancement effect.

[image]

[image]

Terpenes

Terpenes are a class of organic compounds found in essential oils and have been employed as fragrances, flavorings and medicines. A terpene refers to a compound that is based on an isoprene unit (C₅H₈) and can be classified based on the number of isoprenoid units that they contain. For example, a monoterpene consists of two isoprene units (C10), sesquiterpenes have three (C15) and diterpenes have four (C20). A commonly used terpene is menthol, which has been incorporated into inhalation and emollient preparations.

A variety of terpenes, including 1,8-cineole were screened for their ability to enhance the penetration of DFMO through hamster skin. As shown in Table 3 several of these agents at a concentration of 10% in the formulation increased skin penetration of DFMO, in vitro, with the sesquiterpene, nerolidol (cis-3,7,11-trimethyl-1,6,10-dodecatrien-3-ol), producing about a 3-fold enhancement.

TABLE 3
Enhancement of DFMO Penetration through Hamster Skin by
Terpenes (10%) in Hydroalcoholic Formulation 1

| Terpene | Fold Enhancement |
|---|---|
| Nerolidol | 3.03 ± .69 |
| Menthone | 1.99 ± .40 |
| Cineole | 1.91 ± .51 |
| Terpineol | 1.44 ± .20 |
| D-Limonene | 1.36 ± .11 |
| Linalool | 1.29 ± .13 |
| Carvacrol | 1.02 ± .11 |

Nonionic Surfactants

The polyoxyethylene sorbitans or Tweens were also evaluated for effects on DFMO penetration. Shown in Table 4 are the results of the effects of Tween on skin penetration enhancement, again utilizing hamster skin.

TABLE 4
Enhancement of DFMO Penetration though Hamster Skin with Tween
Derivatives (5%) Incorporated into the Hydroalcoholic Formulation 1.

| Compound | Fold Enhancement |
|---|---|
| Tween-40 | 3.07 ± 1.65 |
| Tween-20 | 1.54 ± .47 |
| Tween-60 | 1.09 ± .20 |
| Tween-80 | 0.502 ± .14 |

*with Tween-80 about 50% reduction in penetration was noted, and Tween-60 had nearly no effect.

SEPA

An experiment was conducted to test the diffusion of DFMO through hamster skin after topical application in the cream formulation-I or the cream containing 10% SEPA. The results, shown in FIG. 3, indicates that SEPA (2-n-nonyl-1,3-dioxolane) can increase DFMO permeation about 3-fold from a cream carrier vehicle.

[image]

Film Forming Agents

Film forming agents were investigated based on the hypothesis that when the formulation or vehicle evaporates from the surface of the skin penetration through the skin diminishes. Therefore, by reducing the rate of evaporation of the formulation, it would be possible to prolong the duration of DFMO penetration from a given topical application. Two film-formers that can be employed in topical formulations for sunscreens, lotions, creams and a variety were tested. One of these chemicals, Dermacryl-LT is a high molecular weight carboxylated acrylic copolymer. Methocel, derived from a family of methylcellulose ethers are incorporated into topical products to impart viscosity buildup, also was evaluated in our model system. When 1% Methocel was incorporated into the hydroalcoholic formulation-1, a 4-fold enhancement in DFMO penetration was demonstrated as shown in Table 5.

TABLE 5
Enhancement of DFMO penetration through
Hamster Skin with 1% Methocel.

| | % Applied Dose | | |
|---|---|---|---|
| | Hydroalcoholic | Hydroalcoholic | |
| | Formulation 1 | Formulation 1 with | Fold Enhancement |
| Time (hrs) | (Control) | Methocel | (Methocel/Control) |
| 2 | 0.08 ± .03 | 0.32 ± .10 | 4.0 |
| 6 | 0.28 ± .14 | 1.05 ± .18 | 3.75 |

Dipropylene Glycol Dimethylether

DFMO skin penetration was assessed using a modified protocol of the Franz diffusion assay. In this experiment either the SP33 formulation (without DFMO) or the SP33 formulation prepared with dipropylene glycol dimethylether (DPGDME) again with out DFMO was applied to the skin for 30 or minutes. The formulations were then removed from the skins' surfaces, which were dried with a cotton swab. The hydroalcoholic formulation 1—containing 1% DFMO with radiotracer ¹⁴C-DFMO—was applied to the skin and gently spread over the surface with a glass, stirring rod. Aliquots of the receptor fluid were removed at 3, 6 and 24 hours after DFMO application and penetration was determined using liquid scintillation. As shown in Table 6 enhancement of DFMO penetration through the skin occurred when dipropylene glycol dimethyl ester was substituted for dipropylene glycol. Increases in the amount of DFMO skin penetration at 3 and 24 hours were 4.64-fold greater and 3.02-fold greater, respectively, for the formulation prepared with dipropylene glycol dimethyl ether.

TABLE 6
Skin penetration enhancement of DFMO with dipropylene glycol
dimethyl ester (DPGDME) substituted for dipropylene glycol in
Hydroalcoholic Formulation 1.

| | % Applied Dose | | |
|---|---|---|---|
| Time (hrs) | HA* | DPGDME | Fold-Enhancement |
| 3 | 1.66 ± .23 | 7.71 ± 3.1 | 4.64 |
| 6 | 2.43 ± .32 | 9.55 ± 3.68 | 3.93 |
| 24 | 3.59 ± .42 | 10.85 ± 3.8 | 3.02 |

*HA: hydro-alcoholic formulation-I

Cetiol

Cetiol (dicaprylyl ether) addition to the cream formulation 1 was tested independently for it ability increase skin penetration and the results show about a 2-fold enhancement in skin penetration (Table 7).

TABLE 7
Enhancement of DFMO Skin Penetration Rate by Cetiol

| | Rate of Skin Penetration | | | |
|---|---|---|---|---|
| Time Range | Control | Cetiol | % Increase | p value |
| 2-6 hr | 0.07 ± .01 | 0.13 ± .02 | 209 ± 56 | 0.03 |
| 2-24 hr | 0.06 ± .02 | 0.11 ± .01 | 203 ± 33 | 0.006 |

Rate is expressed as % applied dose/hour×cm²; ±values represent sem; p values were determined using a paired t test. DFMO concentration was 15% in both formulations.

Capric/Caprylic Triglyceride (Captex-300)

As shown by the data in Table 8, Captex-300 inclusion into the hydroalcoholic formulation 1 at a final concentration of 5% gave rise to an increase in DFMO skin penetration, particularly at the 2 and 6 hour sampling time-points.

TABLE 8
DFMO Skin Penetration Enhancement by Capric/Caprylic Triglyceride

| | % Applied Dose | | Fold-Enhancement |
|---|---|---|---|
| Time (hrs) | Control | Captex 5% | Captex/SP33 |
| 2 | 0.86 ± .38 | 3.23 ± .96 | 3.76 |
| 6 | 2.97 ± 1.2 | 8.13 ± 3.4 | 2.73 |
| 24 | 7.88 ± 3.01 | 11.6 ± 3.6 | 1.47 |

Other enhancers that were evaluated included Procetyl-20 (Croda), which is a combination of propylene glycol and Brij-58, isopropyl myristate (IPM), which is used in many pharmaceutical and cosmetic preparations and marketed as estergel, and isostearyl isostearate, a compound similar to isopropyl myristate. All of these agents significantly increased the penetration of DFMO through the skin as shown in Tables 9 and 10.

TABLE 9
Enhancement of DFMO Skin Penetration with Procetyl-20, SEPA and
Isopropyl Myristate as determined using in vitro Assay Protocol #2.

| | % Applied Dose | | Fold-Enhancement |
|---|---|---|---|
| Time (hrs) | 6 Hours | 24 Hours | Enhancer/Control |
| Control* | 2.59 ± .35 | 6.85 ± .79 | — |
| Procetyl 20% | 12 ± 3 | 27 ± 3.4 | 3.94 |
| IPM 5% | 40 ± 6 | 46 ± 6 | 6.71 |

*Control was the hydroalcoholic formulation 1.

TABLE 10
Enhancement of DFMO Skin Penetration with Isostearyl Isostearate
as determined using in vitro Assay Protocol #2.

| | % Applied Dose | | |
|---|---|---|---|
| | | Isostearyl | Fold-Enhancement |
| Time (hrs) | Control* | Isosterate 10% | ISIS/Control |
| 6 | 1.98 ± .26 | 9.7 ± 1.6 | 4.90 |
| 24 | 7.34 ± 1.8 | 23 ± 3.0 | 3.13 |

*Control was the hydroalcoholic formulation 1.

Lauryl Alcohol

Lauryl alcohol produced an increase in DFMO penetration when included in the hydroalcoholic formulation 1 at a concentration of 10% as shown in Table 11. The results suggest about a 1.5-fold increase in skin penetration.

TABLE 11
Enhancement of DFMO Skin Penetration by Lauryl Alcohol

| | % Applied Dose | | Fold Enhancement |
|---|---|---|---|
| Time (hours) | Control | Lauryl Alcohol | Lauryl Alcohol/Control |
| 2 | 0.25 ± .05 | 0.31 ± .03 | 1.24 |
| 6 | 0.36 ± .06 | 0.55 ± .10 | 1.53 |
| 24 | 0.59 ± .11 | 1.01 ± .21 | 1.71 |

*Control was hydroalcoholic formulation 1.

Triacetin

Glyceryl triacetate (triacetin) was demonstrated to moderately increase DFMO penetration through the skin as shown in Table 12 where a 1.7 to 2-fold increase was demonstrated.

TABLE 12
Enhancement of DFMO Skin Penetration by Triacetin

| | % Applied Dose | | Fold-Enhancement |
|---|---|---|---|
| Time (hrs) | Control* | Triacetin 10% | Triacetin/SP106 |
| 2 | 0.46 ± .02 | 0.79 ± .16 | 1.72 |
| 6 | 0.61 ± .04 | 1.35 ± .26 | 2.21 |
| 24 | 2.00 ± .10 | 3.66 ± .64 | 1.83 |

*Control vehicle was cream formulation 1

1-Dodecyl-2-pyrrolidanone

Up to a 5-fold increase in skin penetration by DFMO was generated with the inclusion of 1-dodecyl-2-pyrrolidanone (DDP) into the cream base Formulation-1 at a final concentration of 10% as described in Table 13.

TABLE 13
Enhancement of DFMO Skin Penetration by 1-Dodecyl-2-pyrrolidanone

| | % Applied Dose | | Fold-Enhancement |
|---|---|---|---|
| Time (hrs) | Control* | DDP | DDP/Control |
| 2 | 0.36 ± .25 | 0.15 ± .01 | 0.41 |
| 4 | 0.57 ± .48 | 0.52 ± .08 | 0.91 |
| 6 | 0.63 ± .59 | 1.26 ± .24 | 2.00 |
| 24 | 1.83 ± 1.17 | 9.31 ± 1.38 | 5.11 |

*Control vehicle was cream formulation 1.

Monocaprylate/Caprate (Estol 3601)

Estol 3601 inclusion into the cream formulation provided an increase in DFMO penetratin through the skin as shown in Table 14. The results suggest that a 3-fold increase in skin penetration may be achieved with Estol 3601.

TABLE 14
Enhancement of DFMO Skin Penetration by Estol 3601

| | % Applied Dose | | Fold-Enhancement |
|---|---|---|---|
| Time (hrs) | Control* | Estol 3601 | Estol 3601/Control |
| 2 | 0.36 ± .25 | 0.47 ± .12 | 1.31 |
| 4 | 0.57 ± .48 | 1.05 ± .23 | 1.81 |
| 6 | 0.63 ± .59 | 1.68 ± .33 | 2.67 |
| 24 | 1.83 ± 1.17 | 5.58 ± .82 | 3.05 |

*Control vehicle was cream formulation 1.

The hydro-alcoholic DFMO carrier can be prepared by mixing water (10-60%) with the component alcohols (40-90%). The alcohols can be selected from ethanol, propylene glycol, dipropylene glycol and benzyl alcohol, either added individually, or as a combination thereof. In addition, 1-5% of propylene carbonate can be added to the base hydro-alcoholic vehicle. DFMO, 1-15%, is either dissolved in water, thus replacing the equivalent amount of water from the formulation, or is solubilized in the final vehicle composition such that it results in a proportional decrease in all other vehicle components. The water, alcohols, DFMO, and propylene carbonate levels can be adjusted to achieve a stable formulation in which all components are fully solubilized.

The cream or lotion DFMO formulation can be prepared by first dissolving desired amounts of DFMO (1-15%) in water, which typically is 50-70% in the final cream, then adding emulsifying, co-emulsifying, and emulsion stabilizing agents along with the oil components that need to be emulsified in the formulation. Examples of these are found in Table 1. The components are then sheared to provide an emulsion of desired viscosity. Preservatives, emollients, skin soothing agents, thickening agents, and other components to provide a desired skin feel can be added to the formulation before the shearing process.

### EXAMPLES

Examples of formulations that can be used to provide an increase in DFMO skin penetration are described as follows:

| INCI Name | w/w (%) |
|---|---|
| Example #1 (Cream) | |
| Water | 64.30 |
| DFMO | 15.00 |
| Xanthan gum | 0.20 |
| DC9506^{a} | 2.50 |
| Propylene Glycol | 4.00 |
| Laureth-4 (Brij 30) | 2.00 |
| Ceteareth-20 | 1.00 |
| PEG 100 Stearate + Glyceryl stearate | 2.00 |
| DC200^{b} | 2.00 |
| Cetyl Octanoate | 3.00 |
| Cetyl Alcohol | 2.00 |
| Decyl Alcohol | 1.00 |
| Germaben II^{c} | 1.00 |
| Total | 100.00 |
| ^{a}dimethicone/vinyl dimethicone crosspolymer (Dow Corning, MI); | |
| ^{b}dimethicone (Dow Corning, MI); | |
| ^{c}propylene glycol, Diazolidinyl Urea, methylparaben and propylparaben (Sutton Laboratories, NJ). | |
| Example #2 (Cream) | |
| DI Water | 66.95 |
| DFMO | 15.00 |
| Sepigel 305^{a} | 2.00 |
| DC9040 | 4.50 |
| Cetyl Phosphate | 1.15 |
| DC 5225Cb | 0.50 |
| Brij 72 | 0.25 |
| Aminomethyl Propanol | 0.65 |
| Cetyl Octanoate | 5.00 |
| Pantothenyl Ethyl Ether | 1.00 |
| Cetearyl Alcohol | 2.00 |
| Phenonip^{c} | 1.00 |
| Total | 100.00 |
| ^{a}polyacrylamide, C13-14 isoparaffin and laureth-7 (Seppic, Inc., NJ); | |
| ^{b}Cyclopentasiloxane and dimethicone copolyol (Dow Corning, MI); | |
| ^{c}phenoxyethanol, methyl-, ethyl-, propyl-, butyl- and isobutylparabens (Nipa Inc., DE). | |
| Example #3 (Cream) | |
| DI Water | 60.00 |
| DFMO | 15.00 |
| Sepigel 305a | 2.50 |
| Glyceryl stearate + PEG 100 Stearate | 4.00 |
| Isostearyl Palmitate | 3.00 |
| Ethoxydiglycol | 3.00 |
| Oleic Acid | 2.00 |
| Protaderm HA^{b} | 3.00 |
| Polysorbate (Tween)-40 | 0.50 |
| Glycerin | 3.00 |
| DC556^{c} | 3.00 |
| Germaben II^{d} | 1.00 |
| Total | 100.00 |
| ^{a}polyacrylamide, C13-14 isoparaffin and laureth-7 (Seppic, Inc., NJ); | |
| ^{b}ethoxydiglycol, PEG-7 glyceryl cocoate, salicylic acid, hydroxylauric acid, PPG-12/SMDI copolymer and glycereth-7 (Protameen Chemicals Inc., NJ); | |
| ^{c}phenyl trimethicone (Dow Corning, MI); | |
| ^{d}propylene glycol, Diazolidinyl Urea, methylparaben and propylparaben (Sutton Laboratories, NJ). | |
| Example #4 (Cream) | |
| DI Water | 60.00 |
| DFMO | 15.00 |
| Glyceryl stearate + PEG 100 Stearate | 4.00 |
| Cetearyl Alcohol, Dicetyl Phosphate and Ceteth- | 5.00 |
| 10 phosphate | |
| Caprylic/Capric Triglyceride | 5.00 |
| Lipidure PMB^{a} | 3.00 |
| Advanced Moisture Complex^{b} | 5.00 |
| Cetyl Alcohol | 2.00 |
| Germaben II^{c} | 1.00 |
| Total | 100.00 |
| ^{a}polyquartinium-51 (Collaborative Labs, NY); | |
| ^{b}Glycerin and water and sodium PCA and urea and trehalose and polyqauternium-51 and sodium hyaluronate (Collaborative Labs, NY); | |
| ^{c}propylene glycol, Diazolidinyl Urea, methylparaben and propylparaben (Sutton Laboratories, NJ). | |
| Example #5 (Cream) | |
| DI Water | 61.01 |
| DFMO | 15.00 |
| Mineral oil | 1.89 |
| Glyceryl stearate | 3.60 |
| PEG 100 Stearate | 3.48 |
| Cetearyl Alcohol | 2.59 |
| Ceteareth-20 | 2.13 |
| Dimethicone, 100 ct | 0.48 |
| Lipidure PMB^{a} | 3.00 |
| Advanced Moisture Complex^{b} | 5.00 |
| Stearyl alcohol | 1.42 |
| Phenoxyethanol | 0.29 |
| Methylparaben | 0.08 |
| Propylparaben | 0.03 |
| Total | 100.00 |
| ^{a}polyquartinium-51 (Collaborative Labs, NY); | |
| ^{b}Glycerin and water and sodium PCA and urea and trehalose and polyqauternium-51 and sodium hyaluronate (Collaborative Labs, NY). | |
| Example #6 (Cream) | |
| DI Water | 67.01 |
| DFMO | 15.00 |
| Mineral oil | 1.89 |
| Glyceryl stearate | 3.60 |
| PEG 100 Stearate | 3.48 |
| Cetearyl Alcohol | 2.59 |
| Oleth-20 | 2.13 |
| Dimethicone, 100 ct | 0.48 |
| Decanol | 2.00 |
| Stearyl alcohol | 1.42 |
| Preservative | 0.400 |
| Total | 100.00 |
| Example #7 (Cream) | |
| DI Water | 65.01 |
| DFMO | 15.00 |
| Mineral oil | 1.89 |
| Glyceryl stearate | 3.60 |
| PEG 100 Stearate | 3.48 |
| Cetearyl Alcohol | 2.59 |
| Ceteareth-20 | 2.13 |
| Oleth-20 | 2.00 |
| Dimethicone, 100 ct | 0.48 |
| Decanol | 2.00 |
| Stearyl alcohol | 1.42 |
| Preservative | 0.400 |
| Total | 100.00 |
| Example #8 (Cream) | |
| DI Water | 67.01 |
| DFMO | 15.00 |
| Mineral oil | 1.89 |
| Glyceryl stearate | 3.60 |
| PEG 100 Stearate | 3.48 |
| Oleth-20 | 2.13 |
| Oleyl Alcohol | 2.59 |
| Dimethicone, 100 ct | 0.48 |
| Stearyl alcohol | 1.42 |
| Oleic Acid | 2.00 |
| Phenoxyethanol | 0.29 |
| Methylparaben | 0.08 |
| Propylparaben | 0.03 |
| Total | 100.00 |
| Example #9 (Cream) | |
| DI Water | 65.01 |
| DFMO | 15.00 |
| Glyceryl stearate | 3.60 |
| PEG 100 Stearate | 3.48 |
| Oleth-20 | 2.13 |
| Tween-81 | 2.00 |
| Oleyl Alcohol | 2.59 |
| Caprylic/Capric Triglyceride | 1.89 |
| Dimethicone, 100 ct | 0.48 |
| Stearyl alcohol | 1.42 |
| Glycolic Acid | 2.00 |
| Preservative | 0.400 |
| Total | 100.00 |
| Example #10 (Cream) | |
| DI Water | 65.01 |
| DFMO | 15.00 |
| Mineral oil | 1.89 |
| Cromois HYA^{a} | 2.00 |
| Glyceryl stearate | 3.60 |
| PEG 100 Stearate | 3.48 |
| Oleth-20 | 2.13 |
| Cetearyl Alcohol | 2.59 |
| Dimethicone, 100 ct | 0.48 |
| Stearyl alcohol | 1.42 |
| Oleic Acid | 2.00 |
| Phenoxyethanol | 0.29 |
| Methylparaben | 0.08 |
| Propylparaben | 0.03 |
| Total | 100.00 |
| ^{a}hydrolyzed collagen and hyaluronic acid (Croda Oleochemicals, UK) | |
| Example #11 (Cream) | |
| DI Water | 67.01 |
| DFMO | 15.00 |
| Mineral oil | 1.89 |
| Lanolin Alcohol (Super Hartolan) | 2.00 |
| Glyceryl stearate | 3.60 |
| PEG 100 Stearate | 3.48 |
| Ceteareth-20 | 2.13 |
| Oleyl Alcohol | 2.59 |
| Dimethicone, 100 ct | 0.48 |
| Stearyl alcohol | 1.42 |
| Phenoxyethanol | 0.29 |
| Methylparaben | 0.08 |
| Propylparaben | 0.03 |
| Total | 100.00 |
| Example #12 (Cream) | |
| Primary Emulsion | |
| DI Water | 68.00 |
| Arlacel P135^{a} | 2.00 |
| Arlamol Eb | 7.50 |
| Arlamol HDc | 15.00 |
| Cetiol OEd | 7.00 |
| Germaben II^{e} | 0.50 |
| Total | 100.00 |
| Secondary Emulsion | |
| Primary Emulsion | 50.00 |
| DI Water | 44.00 |
| Xanthan Gum | 0.50 |
| Arlatone 2121^{f} | 5.00 |
| Germaben IIe | 0.50 |
| Total | 100.00 |
| ^{a}PEG-30 dipolyhydroxystearate (Uniqema Americas, NJ); | |
| ^{b}PPG-15 stearyl ether (Uniqema Americas, NJ); | |
| ^{c}isohexadecane (Uniqema Americas, NJ); | |
| ^{d}dicaprylyl ether (Cognis Care Chemicals, PA); | |
| ^{e}propylene glycol, Diazolidinyl Urea, methylparaben and propylparaben (Sutton Laboratories, NJ); | |
| ^{f}sorbitan stearate and sucrose cocoate (Uniqema Americas, NJ). | |
| Example #13 (Cream) | |
| Water | 65.30 |
| DFMO | 15.00 |
| Xanthan gum | 0.20 |
| DC9506^{a} | 2.50 |
| Propylene Glycol | 4.00 |
| Laureth-4 (Brij 30) | 2.00 |
| Ceteareth-20 | 1.00 |
| Glyceryl stearate + PEG 100 Stearate | 2.00 |
| DC200^{b} | 2.00 |
| Cetyl Octanoate | 3.00 |
| Cetyl Alcohol | 2.00 |
| Germaben II^{c} | 1.00 |
| Total | 100.00 |
| ^{a}dimethicone/vinyl dimethicone crosspolymer (Dow Corning, MI); | |
| ^{b}dimethicone (Dow Corning, MI); | |
| ^{c}propylene glycol, Diazolidinyl Urea, methylparaben and propylparaben (Sutton Laboratories, NJ). | |
| Example #14 (hydro-alcoholic) | |
| Water | 53.00 |
| DFMO | 10.00 |
| Ethanol | 16.00 |
| Propylene Glycol | 5.00 |
| Dipropylene Glycol | 5.00 |
| Benzyl Alcohol | 400 |
| Propylene Carbonate | 2.00 |
| Captex-300^{a} | 5.00 |
| Total | 100.00 |
| ^{a}caprylic/capric triglyceride (Abitec Corp., OH). | |
| Example 15 (hydro-alcoholic) | |
| Water | 58.00 |
| DFMO | 10.00 |
| Ethanol | 16.00 |
| Propylene Glycol | 5.00 |
| Dipropylene Glycol dimethyl ether | 5.00 |
| Benzyl Alcohol | 4.00 |
| Propylene Carbonate | 2.00 |
| Total | 100.00 |
| Example 16 (cream) | |
| Water | 70 |
| Glyceryl Stearate | 4 |
| PEG-100 | 4 |
| Cetearyl Alcohol | 3 |
| Ceteareth-20 | 2.5 |
| Mineral Oil | 2 |
| Stearyl Alcohol | 2 |
| Dimethicone | 0.5 |
| Preservatives | 0.43 |
| 1-Dodecyl-2-pyrrolidanone | 1-10 |
| Total | 100.00 |
| DFMO (1-15%) is added to the example 16 formulation and mixed until solubilized | |
| Example 17 (cream) | |
| Water | 70-80 |
| Glyceryl Stearate | 4 |
| PEG-100 | 4 |
| Cetearyl Alcohol | 3 |
| Ceteareth-20 | 2.5 |
| Mineral Oil | 2 |
| Stearyl Alcohol | 2 |
| Dimethicone | 0.5 |
| Preservatives | 0.43 |
| Monocaprylate/Caprate (Estol 3601, Uniquema, | 1-10 |
| NJ) | |
| Total | 100.00 |
| DFMO (1-15%) is added to the example 17 formulation and mixed until solubilized | |
| Example 18 (cream) | |
| Water | 70-80 |
| Glyceryl Stearate | 4 |
| PEG-100 | 4 |
| Cetearyl Alcohol | 3 |
| Ceteareth-20 | 2.5 |
| Mineral Oil | 2 |
| Stearyl Alcohol | 2 |
| Dimethicone | 0.5 |
| Preservatives | 0.43 |
| cis Fatty Acids | 1-10 |
| Total | 100.00 |
| DFMO (1-15%) is added to the example 18 formulation and mixed until solubilized | |
| Example 19 (ceam) | |
| Water | 70-80% |
| Glyceryl Stearate | 4 |
| PEG-100 | 4 |
| Cetearyl Alcohol | 3 |
| Ceteareth-20 | 2.5 |
| Mineral Oil | 2 |
| Stearyl Alcohol | 2 |
| Dimethicone | 0.5 |
| Preservatives | 0.43 |
| Terpenes | 1-10 |
| Total | 100.00 |
| DFMO (1-15%) is added to the example 19 formulation and mixed until solubilized | |
| Example 20 (cream) | |
| Water | 70-80% |
| Glyceryl Stearate | 4 |
| PEG-100 | 4 |
| Cetearyl Alcohol | 3 |
| Ceteareth-20 | 2.5 |
| Mineral Oil | 2 |
| Stearyl Alcohol | 2 |
| Dimethicone | 0.5 |
| Preservatives | 0.43 |
| Polyoxyethylene sorbitans (tween) | 1-10 |
| Total | 100.00 |

DFMO (1-15%) is added to the example 20 formulation and mixed until solubilized
Other embodiments are within the scope of the following claims.

What is claimed is:

1. A method of reducing human hair growth, comprising selecting an area of skin from which reduced hair growth is desired, and applying to the area of skin, in an amount effective to reduce hair growth, a composition including alpha-difluoromethylornithine and a dermatologically acceptable vehicle comprising a cis-fatty acid.

2. The method of claim 1, wherein the vehicle includes from 0.1% to 20% by weight of the cis-fatty acid.

3. The method of claim 1, wherein the vehicle includes from 1% to 10% by weight of the cis-fatty acid.

4. The method of claim 1, wherein the cis-fatty acid includes from 8 to 30 carbon atoms.

5. The method of claim 1, wherein the cis-fatty acid is selected from the group consisting of erucic acid, palmitoleic acid, petroselenic acid, lauric acid, and oleic acid.

6. The method of claim 1, wherein the composition includes from 5% to 20% by weight alpha-difluoromethylornithine.

7. The method of claim 1, wherein the alpha-difluoromethylomithine comprises at least about 80% of L-alpha-difluoromethylornithine.

8. The method of claim 1, wherein the alpha-difluoromethylomithine comprises at least about 95% of L-alpha-difluoromethylomithine.

9. The method of claim 1, wherein the area of skin is on the face, legs, or axilla of a human.

10. A composition for topical application to the skin, comprising alpha-difluoromethylomithine in an amount effective to reduce hair growth and a dermatologically acceptable vehicle comprising a cis-fatty acid.

11. A method of reducing human hair growth, comprising
selecting an area of skin from which reduced hair growth is desired, and
applying to the area of skin, in an amount effective to reduce hair growth, a composition including alpha-difluoromethylomithine and a dermatologically acceptable vehicle comprising a fatty alcohol.

12. The method of claim 11, wherein the vehicle includes from 0.1% to 20% by weight of the fatty alcohol.

13. The method of claim 11, wherein the vehicle includes from 1% to 10% by weight of the fatty alcohol.

14. The method of claim 11, wherein the fatty alcohol includes from 8 to 30 carbon atoms.

15. The method of claim 11, wherein the fatty alcohol is selected from the group consisting of decanol, oleyl alcohol, and lauryl alcohol.

16. The method of claim 11, wherein the composition includes from 5% to 20% by weight α-difluoromethylornithine.

17. The method of claim 11, wherein the alpha-difluoromethylornithine comprises at least about 80% of L-alpha-difluoromethylomithine.

18. The method of claim 11, wherein the alpha-difluoromethylomithine comprises at least about 95% of L-alpha-difluoromethylomithine.

19. The method of claim 11, wherein the area of skin is on the face, legs, or axilla of a human.

20. A composition for topical application to the skin, comprising alpha-difluoromethylornithine in an amount effective to reduce hair growth and a dermatologically acceptable vehicle comprising a fatty alcohol

21. A method of reducing hair growth, comprising
selecting an area of skin from which reduced hair growth is desired, and
applying to the area of skin, in an amount effective to reduce hair growth, a composition including alpha-difluoromethylomithine and a dermatologically acceptable vehicle comprising a fatty acid ester.

22. The method of claim 21, wherein the vehicle includes from 0.1% to 20% by weight of the fatty acid ester.

23. The method of claim 21, wherein the vehicle includes from 1% to 10% by weight of the fatty acid ester.

24. The method of claim 21, wherein the fatty acid ester includes from 12 to 60 carbon atoms.

25. The method of claim 21, wherein the fatty acid ester is selected from the group consisting of dodecyl N,N,-dimethylamino acetate, isopropyl isostearate, ethyl acetate, isostearyl isostearate, isopropyl myristate, and oleyl oleate.

26. The method of claim 21, wherein the composition includes from 5% to 20% by weight α-difluoromethylomithine.

27. The method of claim 21, wherein the alpha-difluoromethylomithine comprises at least about 80% of L-alpha-difluoromethylomithine.

28. The method of claim 1, wherein the alpha-difluoromethylomithine comprises at least about 95% of L-alpha-difluoromethylornithine.

29. The method of claim 1, wherein the area of skin is on the face, legs, or axilla of a human.

30. A composition for topical application to the skin, comprising alpha-difluoromethylornithine in an amount effective to reduce hair growth and a dermatologically acceptable vehicle comprising a fatty acid ester.

31. A method of reducing hair growth, comprising
selecting an area of skin from which reduced hair growth is desired, and
applying to the area of skin, in an amount effective to reduce hair growth, a composition including alpha-difluoromethylornithine and a dermatologically acceptable vehicle comprising a terpene.

32. The method of claim 31, wherein the vehicle includes from 0.1% to 20% by weight of the terpene.

33. The method of claim 21, wherein the vehicle includes from 1% to 10% by weight of the terpene.

34. The method of claim 21, wherein the terpene includes from 10 to 20 carbon atoms.

35. The method of claim 31, wherein the terpene is selected from the group consisting of nerolidol, menthone, 1,8-cineole, terpineol, D-limonene, linalool and carvacrol

36. The method of claim 31, wherein the composition includes from 5% to 20% by weight α-difluoromethylornithine.

37. The method of claim 31, wherein the alpha-difluoromethylomithine comprises at least about 80% of L-alpha-difluoromethylomithine.

38. The method of claim 31, wherein the alpha-difluoromethylomithine comprises at least about 95% of L-alpha-difluoromethylomithine.

39. The method of claim 31, wherein the area of skin is on the face, legs, or axilla of a human.

40. A composition for topical application to the skin, comprising alpha-difluoromethylornithine in an amount effective to reduce hair growth and a dermatologically acceptable vehicle a comprising a terpene.

41. A method of reducing human hair growth, comprising
selecting an area of skin from which reduced hair growth is desired, and
applying to the area of skin, in an amount effective to reduce hair growth, a composition including alpha-difluoromethylomithine and a dermatologically acceptable vehicle comprising a nonionic surfactant selected from the group consisting of polyoxyethylene sorbitants.

42. The method of claim 41, wherein the vehicle includes from 0.1% to 20% by weight of the nonionic surfactant.

43. The method of claim 41, wherein the vehicle includes from 1% to 10% by weight of the nonionic surfactant.

44. The method of claim 1, wherein the polyoxyethylene sorbitant comprises a polyoxyethylene (2-150) sorbatan fatty acid (C₆-C₃₀) ester.

45. The method of claim 41, wherein the polyoxyethylene sorbitants is selected from the group consisting of Tween-20, Tween 40, and Tween 60.

46. The method of claim 41, wherein the composition includes from 5% to 20% by weight α-difluoromethylornithine.

47. The method of claim 41, wherein the alpha-difluoromethylomithine comprises at least about 80% of L-alpha-difluoromethylomithine.

48. The method of claim 41, wherein the alpha-difluoromethylornithine comprises at least about 95% of L-alpha-difluoromethylomithine.

49. The method of claim 41, wherein the area of skin is on the face, legs, and axilla of a human.

50. A composition for topical application to the skin, comprising alpha-difluoromethylornithine in an amount effective to reduce hair growth and a dermatologically acceptable vehicle comprising a nonionic surfactant selected from the group consisting of polyoxyethylene sorbitants.

51. A method of reducing human hair growth, comprising
selecting an area of skin from which reduced hair growth is desired, and
applying to the area of skin, in an amount effective to reduce hair growth, a composition including alpha-difluoromethylomithine and a dermatologically acceptable vehicle comprising a film forming agent.

52. The method of claim 51, wherein the vehicle includes from 0.1% to 20% by weight of the film forming agent.

53. The method of claim 51, wherein the vehicle includes from 1% to 10% by weight of the film forming agent.

54. The method of claim 51, the filming forming agent is a methyl cellulose ether.

55. The method of claim 51, wherein the methyl cellulose ether is methocel.

56. The method of claim 51, wherein the film forming agent is carboxylated acrylic copolymer.

57. The method of claim 56, wherein the carboxylated acrylic copolymer is Dermacyl-LT.

58. A method of reducing human hair growth, comprising
selecting an area of skin from which reduced hair growth is desired, and
applying to the area of skin, in an amount effective to reduce hair growth, a composition including alpha-difluoromethylomithine and a dermatologically acceptable vehicle comprising a preferred chemical agent selected from 2,n-nonyl-1,3-dioxolane (SEPA), dipropylene glycol dimethyl ether, dicaprylyl ether (Cetiol), capric/caprylic triglyceride, monocaprylate/caprate, glyceryl triacetate (triacetin), and 1-dodecyl-2-pyrrolidanone.

59. The method of claim 58, wherein the vehicle includes from 0.1% to 20% by weight of the preferred chemical agent of claim 58.

60. The method of claim 58, wherein the vehicle includes from 1% to 10% by weight of the preferred chemical agent of claim 58.

61. The method of claim 4, wherein the cis-fatty acid includes from 8 to 12 carbon atoms.

62. The method of claim 14, wherein the fatty alcohol includes from 12 to 18 carbon atoms.

63. The method of claim 24, wherein the fatty acid ester includes from 16 to 36 carbon atoms.

64. The method of claim 31, wherein the terpene includes one isoprene unit (C5), one monoterpene unit (C10), or 2 to 10 isoprene units (C₁₀ to C₅₀).

65. The method of claim 44, wherein the polyoxyethylene sorbitan comprises a polyethylene (20-80) sorbitan fatty acid (C₁₂ to C₁₈) ester.

66. A composition comprising a vehicle containing 1% to 15% by weight alpha-difluoromethylornithine and 0.5% to 15% of the cis-fatty acid.

67. The composition of claim 66, wherein the composition comprises from 1% to 10% by weight of a cis-fatty acid.

68. A composition comprising a vehicle containing 1% to 15% by weight alpha-difluoromethylornithine and 0.5% to 15% of a terpene.

69. The composition of claim 68, wherein the composition comprises from 1% to 10% by weight of the terpene.

70. A composition comprising a vehicle containing 1% to 15% by weight alpha-difluoromethylornithine and 0.5% to 15% of polyoxyethylene sorbitan.

71. The composition of claim 70, wherein the composition comprises from 1% to 10% by weight of the polyoxyethylene sorbitan.

72. A composition comprising a vehicle containing 1% to 15% by weight alpha-difluoromethylornithine and 0.5% to 15% of an agent selected from the group comprising of 2-n-nonyl-1,3-dioxolane (SEPA), dipropylene glycol dimethylether, cetiol. capric/caprylic triglyceride (Captex-300), procetyl-20, isopropyl myristate, isostearyl isostearate, lauryl alcohol, triacetin, 1-dodecyl-2-pyrrolidanone, and monocaprylate/caprate.

73. The composition of claim 72, wherein the composition comprises from 1% to 10% by weight of the agent.

74. A composition comprising a vehicle containing 1% to 15% by weight alpha-difluoromethylornithine and 0.5% to 15% of a skin film forming agent selected from the group consisting of methyl cellulose ethers and carboxylated acrylic copolymers.

75. The composition of claim 74, wherein the composition comprises from 1% to 10% of the film-forming agent.
